SEQUENCE LISTING (17 sequences)

SEQ ID NO: 1 - protein, 5 aa, Artificial
  A-motif
XXDYS

SEQ ID NO: 2 - protein, 5 aa, Artificial
  B-motif
GXPSG

SEQ ID NO: 3 - protein, 4 aa, Artificial
  C-motif
YGDD

SEQ ID NO: 4 - protein, 5 aa, Artificial
  D-motif
XXYGL

SEQ ID NO: 5 - protein, 10 aa, Artificial
  E-Motif
XXXXFLXRXX

SEQ ID NO: 6 - protein, 520 aa, Norovirus
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALE

SEQ ID NO: 7 - protein, 517 aa, Sapovirus
MKDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEME

SEQ ID NO: 8 - protein, 533 aa, Vesivirus
MKVTTQKYDVTKPDISYKGLICKQLDEIRVIPKGTRLHVSPAHTDDYDECSHQPASLGSGDPRCPKSLTAIVVDSLKPYCEKTDGPPHDILHRVQRMLIDHLSGFVPMNISSEPSMLAAFHKLNHDTSCGPYLGGRKKDHMIGGEPDKPLLDLLSSKWKLATQGIGLPHEYTIGLKDELRPVEKVQEGKRRMIWGCDVGVATVCAAAFKGVSDAITANHQYGPVQVGINMDGPSVEALYQRIRSAAKVFAVDYSKWDSTQSPRVSAASIDILRYFSDRSPIVDSAANTLKSPPIAIFNGVAVKVTSGLPSGMPLTSVINSLNHCLYVGCAILQSLESRNIPVTWNLFSTFDMMTYGDDGVYMFPMMFASVSDQIFANLTAYGLKPTRVDKSVGAIEPIDPESVVFLKRTITRTPHGIRGLLDRGSIIRQFYYIKGENSDDWKTPPKTIDPTSRGQQLWNACLYASQHGPEFYNKVYRLAEKAVEYEELHFEPPSYHSALEHYNNQFNGVDTRSDQIDASVMTDLHCDVFEVLE

SEQ ID NO: 9 - protein, 517 aa, Lagovirus
MTSNFFCGEPIDYRGITAHRLVGAEPRPPVSGTRYAKVPGVPDEYKTGYRPANLGRSDPDSDKSLMNIAVKNLQVYQQEPKLDKVDEFIERAAADVLGYLRFLTKGERQANLNFKAAFNTLDLSTSCGPFVPGKKIDHVKDGVMDQVLAKHLYKCWSVANSGKALHHIYACGLKDELRPLDKVKEGKKRLLWGCDVGVAVCAAAVFHNICYKLKMVARFGPIAVGVDMTSRDVDVIINNLTSKASDFLCLDYSKWDSTMSPCVVRLAIDILADCCEQTELTKSVVLTLKSHPMTILDAMIVQTKRGLPSGMPFTSVINSICHWLLWSAAVYKSCAEIGLHCSNLYEDAPFYTYGDDGVYAMTPMMVSLLPAIIENLRDYGLSPTAADKTEFIDVCPLNKISFLKRTFELTDIGWVSKLDKSSILRQLEWSKTTSRHMVIEETYDLAKEERGVQLEELQVAAAAHGQEFFNFVCRELERQQAYTQFSVYSYDAARKILADRKRVVSVVPDDEFVNVME

SEQ ID NO: 10 - protein, 526 aa, Artificial
  Norovirus RNA polymerase containing His tag
MGGDSKGTYCGAPILGPGSAPKLSTKTKFWRSSTTPLPPGTYEPAYLGGKDPRVKGGPSLQQVMRDQLKPFTEPRGKPPKPSVLEAAKKTIINVLEQTIDPPEKWSFTQACASLDKTTSSGHPHHMRKNDCWNGESFTGKLADQASKANLMFEGGKNMTPVYTGALKDELVKTDKIYGKIKKRLLWGSDLATMIRCARAFGGLMDELKAHCVTLPIRVGMNMNEDGPIIFERHSRYKYHYDADYSRWDSTQQRAVLAAALEIMVKFSSEPHLAQVVAEDLLSPSVVDVGDFKISINEGLPSGVPCTSQWNSIAHWLLTLCALSEVTNLSPDIIQANSLFSFYGDDEIVSTDIKLDPEKLTAKLKEYGLKPTRPDKTEGPLVISEDLNGLTFLRRTVTRDPAGWFGKLEQSSILRQMYWTRGPNHEDPSETMIPHSQRPIQLMSLLGEAALHGPAFYSKISKLVIAELKEGGMDFYVPRQEPMFRWMRFSDLSTWEGDRNLAPSFVNEDGVEVDKLAAALEHHHHHH

SEQ ID NO: 11 - protein, 523 aa, Artificial
  Sapovirus RNA polymerase containing His tag
MKHHHHHHDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEME

SEQ ID NO: 12 - protein, 523 aa, Artificial
  Sapovirus RNA polymerase containing His tag
MKDEFQWKGLPVVKSGLDVGGMPTGTRYHRSPAWPEEQPGETHAPAPFGAGDKRYTFSQTEMLVNGLKPYTEPTAGVPPQLLSRAVTHVRSYIETIIGTHRSPVLTYHQACELLERTTSCGPFVQGLKGDYWDEEQQQYTGVLANHLEQAWDKANKGIAPRNAYKLALKDELRPIEKNKAGKRRLLWGCDAATTLIATAAFKAVATRLQVVTPMTPVAVGINMDSVQMQVMNDSLKGGVLYCLDYSKWDSTQNPAVTAASLAILERFAEPHPIVSCAIEALSSPAEGYVNDIKFVTRGGLPSGMPFTSVVNSINHMIYVAAAILQAYESHNVPYTGNVFQVETVHTYGDDCMYSVCPATASIFHAVLANLTSYGLKPTAADKSDAIKPTNTPVFLKRTFTQTPHGVRALLDITSITRQFYWLKANRTSDPSSPPAFDRQARSAQLENALAYASQHGPVVFDTVRQIAIKTAQGEGLVLVNTNYDQALATYNAWFIGGTVPDPVGHTEGTHKIVFEMEHHHHHH

SEQ ID NO: 13 - protein, 539 aa, Artificial
  Vesivirus RNA polymerase containing His tag
MKVTTQKYDVTKPDISYKGLICKQLDEIRVIPKGTRLHVSPAHTDDYDECSHQPASLGSGDPRCPKSLTAIVVDSLKPYCEKTDGPPHDILHRVQRMLIDHLSGFVPMNISSEPSMLAAFHKLNHDTSCGPYLGGRKKDHMIGGEPDKPLLDLLSSKWKLATQGIGLPHEYTIGLKDELRPVEKVQEGKRRMIWGCDVGVATVCAAAFKGVSDAITANHQYGPVQVGINMDGPSVEALYQRIRSAAKVFAVDYSKWDSTQSPRVSAASIDILRYFSDRSPIVDSAANTLKSPPIAIFNGVAVKVTSGLPSGMPLTSVINSLNHCLYVGCAILQSLESRNIPVTWNLFSTFDMMTYGDDGVYMFPMMFASVSDQIFANLTAYGLKPTRVDKSVGAIEPIDPESVVFLKRTITRTPHGIRGLLDRGSIIRQFYYIKGENSDDWKTPPKTIDPTSRGQQLWNACLYASQHGPEFYNKVYRLAEKAVEYEELHFEPPSYHSALEHYNNQFNGVDTRSDQIDASVMTDLHCDVFEVLEHHHHHH

SEQ ID NO: 14 - protein, 523 aa, Artificial
  Lagovirus RNA Polymerase containing His tag
MTSNFFCGEPIDYRGITAHRLVGAEPRPPVSGTRYAKVPGVPDEYKTGYRPANLGRSDPDSDKSLMNIAVKNLQVYQQEPKLDKVDEFIERAAADVLGYLRFLTKGERQANLNFKAAFNTLDLSTSCGPFVPGKKIDHVKDGVMDQVLAKHLYKCWSVANSGKALHHIYACGLKDELRPLDKVKEGKKRLLWGCDVGVAVCAAAVFHNICYKLKMVARFGPIAVGVDMTSRDVDVIINNLTSKASDFLCLDYSKWDSTMSPCVVRLAIDILADCCEQTELTKSVVLTLKSHPMTILDAMIVQTKRGLPSGMPFTSVINSICHWLLWSAAVYKSCAEIGLHCSNLYEDAPFYTYGDDGVYAMTPMMVSLLPAIIENLRDYGLSPTAADKTEFIDVCPLNKISFLKRTFELTDIGWVSKLDKSSILRQLEWSKTTSRHMVIEETYDLAKEERGVQLEELQVAAAAHGQEFFNFVCRELERQQAYTQFSVYSYDAARKILADRKRVVSVVPDDEFVNVMEHHHHHH

SEQ ID NO: 15 - RNA, 24 nt, Artificial
  ssRNA template
auaccuagaaucugaccaaccccc

SEQ ID NO: 16 - DNA, 24 bp, Artificial
  ssDNA template
atacctagaatctgaccaaccccc

SEQ ID NO: 17 - DNA, 24 bp, Artificial
  ssDNA template having 5 rC at the 3′-end
atacctagaatctgaccaaccccc

(19) United States
(12) Patent Application Publication
(10) Pub. No.: US 2013/0189742 A1
(43) Pub. Date: Jul. 25, 2013

(54) Microwave-Driven RNA Polymerization by RNA Polymerases of Caliciviruses

(71) Applicant: Jacques Rohayem, Dresden (DE)

(72) Inventor: Jacques Rohayem, Dresden (DE)

(21) Appl. No.: 13/825,116

(22) PCT Filed: Sep. 20, 2011
(86) PCT No.: PCT/EP2011/066362

§ 371 Date: Mar. 19, 2013

(30) Foreign Application Priority Data
Sep. 21, 2010 (EP) 10178114.4

(51) Int. Cl. C12P 19/34

(52) U.S. Cl. CPC C12P 19/34
USPC 435/91.3

(57) ABSTRACT

The present invention relates to a method for polymerising a complementary RNA strand on a single-stranded polynucleotide template comprising the step of irradiating a composition containing said template and an RNA polymerase of a virus of the Caliciviridae family under RNA polymerisation conditions in the presence or absence of a primer hybridised to the template, with an effective amount of microwave energy. Further subject matter of the invention relates to a method for transferring one or more ribonucleotides to the 3′ end of a single-stranded polynucleotide template comprising the step of irradiating a composition containing an RNA polymerase of a virus of the Caliciviridae family in the presence of rATP or rGTP or rUTP or rCTP or a modified or labelled analogue thereof with an effective amount of microwave energy.

The present invention relates to a method for polymerising a complementary RNA strand on a single-stranded polynucleotide template comprising the step of irradiating a composition containing said template and an RNA polymerase of a virus of the Caliciviridae family under RNA polymerisation conditions in the presence or absence of a primer hybridised to the template, with an effective amount of microwave energy. Further subject matter of the invention relates to a method for transferring one or more ribonucleotides to the 3′ end of a single-stranded polynucleotide template comprising the step of irradiating a composition containing an RNA polymerase of a virus of the Caliciviridae family in the presence of rATP or rGTP or rUTP or rCTP or a modified or labelled analogue thereof with an effective amount of microwave energy.

RNA-dependent RNA polymerases (in the following denoted as “RNA polymerases”) of viruses of the Caliciviridae family are known to polymerise a complementary RNA strand on an RNA template in the presence or absence of a primer (see WO-A-2007/12329). Using such RNA polymerases, a typical amplification of RNA at usual temperature conditions takes approximately 2 hours.

U.S. Pat. No. 5,350,686 generally claims microwave acceleration of enzyme-catalysed modifications of macromolecules, but factually shows only successful microwave-assisted reactions in the case of restriction enzymes.

U.S. Pat. No. 7,537,917 describes a microwave-assisted PCR amplification of DNA. However, no data whatsoever are presented that factually show a successful DNA polymerisation by DNA polymerases under microwave irradiation.

In particular with regard to DNA amplification using PCR, only the heating (i.e denaturation) step (and not the polymerisation step) within the amplification cycles has been reported to be a step suitable for microwave application; see Fermér et al. (2003) European J. Pharm. Sci. 18, 129-132.

The technical problem underlying the present invention is to provide an improved method of RNA polymerisation on polynucleotide templates.

The solution to the above technical problem is provided by the embodiments of the present invention as described herein and characterised in the claims.

In particular, it has been surprisingly found that the complex reaction of polymerising a complementary RNA strand on a single-stranded polynucleotide template (RNA, DNA, mixed RNA/DNA or mixtures thereof) is feasible and greatly enhanced by microwave irradiation when using RNA polymerases of viruses of the Caliciviridae family but not other RNA polymerases studied in the experiments leading to the present invention.

Thus, the present invention relates to a method for polymerising a complementary RNA strand on a single-stranded polynucleotide template comprising irradiating a composition containing said template and an RNA polymerase of a virus of the Caliciviridae family under RNA polymerisation conditions in the presence or absence of a primer hybridised to the template, to an effective amount of microwave energy.

RNA polymerases of the caliciviruses have (as have other viral RNA-dependent RNA polymerases such as RNA polymerase from poliovirus or Hepatitis C Virus (HCV) which, however, are not applicable to the inventive method; cf. the Examples described below) the following structural features in common: RNA polymerases of the viruses of the Caliciviridae family have a “right hand conformation” and the amino acid sequence of said RNA polymerases comprises a conserved arrangement of the following sequence motifs:

a.
(SEQ ID NO: 1)
XXDYS
b.
(SEQ ID NO: 2)
GXPSG
c.
(SEQ ID NO: 3)
YGDD
d.
(SEQ ID NO: 4)
XXYGL
e.
(SEQ ID NO: 5)
XXXXFLXRXX

with the following meanings:

D: aspartate

Y: tyrosine

S: serine

G: glycine

P: proline

L: leucine

F: phenylalanine

R: arginine

X: any amino acid.

The so-called “right hand conformation” as used herein means that the tertiary structure (conformation) of the RNA polymerase folds like a right hand with finger, palm and thumb, as observed in most template-dependent polymerases.

The sequence motif “XXDYS” (SEQ ID NO: 1) is the so-called A-motif. The A-motif is usually responsible for the discrimination between ribonucleosides and deoxyribonucleosides. The motif “GXPSG” (SEQ ID NO: 2) is the so-called B-motif. The B-motif is conserved within all representatives of the RNA polymerases of the Caliciviridae family. The motif “YGDD” (C-motif, SEQ ID NO: 3) represents the active site of the enzyme. This motif, in particular the first aspartate residue (in bold, YGDD) plays an important role in the coordination of the metal ions during the Mg²⁺/Mn²⁺ dependent catalysis. The motif “XXYGL” (SEQ ID NO: 4) is the so-called D-motif. The D-motif is a feature of template-dependent polymerases. Finally, the “XXXXFLXRXX” motif (E-motif, SEQ ID NO: 5) is a feature of RNA-dependent RNA polymerases which discriminates them from (exclusively) DNA-dependent RNA polymerases.

Preferably, the RNA polymerase is an RNA polymerase of a human and/or non-human pathogenic calicivirus. Especially preferred is an RNA polymerase of a norovirus, sapovirus, vesivirus or lagovirus, for example, an RNA polymerase of the norovirus strain HuCV/NL/Dresden174/1997/GE (GenBank Acc. No AY741811) or an RNA polymerase of the sapovirus strain pJG-Sap01 (GenBank Acc. No AY694184) or an RNA polymerase of the vesivirus strain FCV/Dresden/2006/GE (GenBank Acc. No DQ424892) or an RNA polymerase of the lagovirus strain pJG-RHDV-DD06 (GenBank Acc. No. EF363035.1).

According to especially preferred embodiments of the invention the RNA polymerase is a protein comprising (or having) an amino acid sequence according SEQ ID NO: 6 (norovirus RNA polymerase), SEQ ID NO: 7 (sapovirus RNA polymerase), SEQ ID NO: 8 (vesivirus RNA polymerase) or SEQ ID NO: 9 (lagovirus RNA polymerase). The person skilled in the art is readily capable of preparing such RNA polymerases, for example by recombinant expression using suitable expression vectors and host organisms (cf. WO-A-2007/012329). To facilitate purification of the RNA polymerase after recombinant expression, it is preferred that the RNA polymerase is expressed with a suitable tag (for example GST or (His)₆-tag) at the N- or C-terminus of the corresponding sequence. For example, a histidine tag allows the purification of the protein by affinity chromatography over a nickel or cobalt column in a known fashion. Examples of embodiments of RNA polymerases fused to a histidine tag are the proteins comprising (or having) an amino acid sequence according to SEQ ID NO: 10, SEQ ID NO: 11, SEQ ID NO: 12, SEQ ID NO: 13 or SEQ ID NO: 14. SEQ NO: NO: 10 corresponds to an RNA polymerase of a norovirus having a histidine tag. SEQ ID NO: 11 and SEQ ID NO: 12 correspond to amino acid sequences of an RNA polymerase of a sapovirus having a histidine tag. SEQ ID NO: 13 corresponds to the amino acid sequence of an RNA polymerase of a vesirius having a histidine tag. SEQ ID NO: 14 corresponds to the amino acid sequence of an RNA polymerase of a lagovirus having a histidine tag.

The above-defined RNA polymerase of a calicivirus is capable of synthesizing a complementary RNA strand on a polynucleotide strand of ribonucleotides (i.e. the polynucleotide template consists of or comprises RNA) or deoxyribonucleotides (i.e. the polynucleotide template consists of or comprises DNA). Accordingly, the polynucleotide template may be single-stranded RNA, single-stranded DNA, single-stranded mixed DNA/RNA or a mixture of such species. If the polynucleotide template comprises or consists of DNA, the irradiation with microwaves must be carried out in the presence of a modified GTP, preferably a 2′.modified GTP such as 2′-fluoro-GTP, or α-thio-GTP. The RNA polymerase of a calicivirus synthesises a complementary RNA strand on a single-stranded polynucleotide both by elongation of a primer having a complementary sequence to a partial sequence of the template DNA and by de novo synthesis of a complementary strand in the absence of a primer. However, if the polynucleotide template containing deoxyribonucleotides has a deoxyribonucleotide at its 3′-end (i.e. the last nucleotide at the 3′-end of the single-stranded template) which is not a deoxy-C nucleoteotide (i.e. has a deoxy-T, deoxy-A or deoxy-G nucleotide at its 3′-end), the RNA polymerase of a calicivirus useful in the present invention requires the presence of a primer hybridised to the template for synthesis of an RNA strand complementary to the template. If the polynucleotide template as defined herein consists of or contains one or more deoxyribonucleotides at its 3′-end (i.e. the 3′-end of the template is a DNA segment or only the last nucleotide is a deoxyribonucleotide), it is preferred that the last deoxyribonucleotide at the 3′-end of the template is a dC, more preferred at least the last two, three, four or five deoxyribonucleotides at the 3′-end of the template are dC nucleotides for increasing the efficiency of de novo initiation of RNA synthesis in the absence of a primer.

In case of templates consisting of RNA or of templates in which the region at the 3′-end is composed of ribonucleotides, the polymerisation in the inventive microwave irradiation method requires the presence of a corresponding primer, if the template is polyadenylated, polyguanylated or polyuridylated. If the template is polycytidylated, RNA synthesis by the calicivirus RNA polymerase is also possible without a primer. In this embodiment it is preferred that elevated rGTP levels (i.e. a surplus of GTP such as, e.g., 2×, 3×, 4× or 5× compared to the other required rNTPs) are present.

The primer, if desired or required, respectively, may be a sequence specific (heteropolymeric) DNA or RNA or mixed DNA/RNA primer or may be a random primer (DNA or RNA or mixed DNA/RNA) or may be a homopolymeric primer such as an oligo-dT-primer or an oligo-U-Primer. The length of the primer is not critical for carrying out the inventive method, but usually oligonucleotide primers having a length of, for example, about 5 to about 25 nt, more preferred about 10 to 20 nt, most preferred about 15 to about 20 nt, are especially useful. More details of the characteristic features of the calicivirus RNA polymerase can be found in WO-A-2007/012329. In contrast to other RNA-dependent RNA polymerases, e.g. RNA-dependent RNA polymerases such as replicases of the Qβ type, the RNA polymerases of the caliciviruses do not require primers having a specific recognition sequence for the polymerase to start RNA synthesis. Thus, a “primer” as used herein is typically a primer not having such recognition sequences, in particular, of RNA polymerases. Furthermore, the calicivirus RNA polymerases are different from usual DNA-dependent RNA polymerases such as T7 RNA polymerase in that they do not require specific promoter sequences to be present in the template.

The single-stranded polynucleotide template may comprise at least a sequence segment of deoxyribonucleotides, i.e. at least a segment of ssDNA, e.g. at least a segment of DNA at the 3′-end of the template. A “segment” in this context means at least 2 or more consecutive deoxyribonucleotides. For example, the polynucleotide template according to the invention may be a single-stranded molecule starting at its 5′-end with ribonucleotides, followed by a “middle” region of deoxyribonucleotides (DNA) and ends (at the 3′-end) again with ribonucleotides. Other examples are species of 5′-RNA-DNA-3′ or 5′-DNA-RNA-3′ or any other polynucleotides having RNA and DNA sequences. Further examples of single-stranded polynucleotide templates according to the invention include species of predominantly ssDNA, but having one to multiple (such as 1, 2, 3, 4, 5, 6, 7, 8, 9 or 10) ribonucleotides at one or both of the 5′-end and/or 3′-end, preferably at the 3′-end. Alternatively, templates of use according to the invention may be predominantly ssRNA, but having one to multiple (such as 1,2,3, 4, 5, 6, 7, 8, 9 or 10) deoxyribonucleotides at one or both of the 5′-end and/or 3′-end, preferably at the 3′-end. Of course, the single-stranded polynucleotide template according to the invention may also consist exclusively of ssDNA or ssRNA.

As mentioned before, polynucleotide templates of the invention having a dA, dT or dG residue at the 3′-end normally require a primer for synthesis of a complementary RNA strand by the RNA polymerase. However, even such polynucleotide sequences not having a C nucleotides at the 3′-end can be efficiently transcribed into RNA by the inventive method without the need of a primer: especially in this case, but not limited thereto, the method may be carried with an initial step of irradiating a composition containing the single-stranded polynucleotide template and the RNA polymerase of a calicivirus as defined above in the presence of rCTP as the only nucleotide with an efficient amount of microwave energy under conditions such that said RNA polymerase adds at least one rC (or more such as 2, 3, 4 or 5 rC) nucleotide to the 3′-end of the template. Thereafter, the thus-produced template having one or more C ribonucleotides at the 3′-end can be introduced to the microwave-driven RNA synthesis by the RNA polymerase as defined above such that said RNA polymerase synthesizes a complementary RNA strand, which step may be carried out in the absence of a primer. It is to be understood, however, that also a primer may be used in this embodiment, for example, if needed to introduce a chosen sequence into the RNA strand to be produced by the RNA polymerase or for other purposes.

In view of the terminal transferase activity of the RNA polymerases of caliciviruses, the present invention provides also a method for transferring one or more ribonucleotides to the 3′ end of a single-stranded polynucleotide template as defined above comprising the step of irradiating a composition containing an RNA polymerase of a virus of the Caliciviridae family as defined above in the presence of rATP, rGTP, rUTP or rCTP or a modified or labelled analogue thereof, with an efficient amount of microwave energy.

According to a preferred embodiment of the method of the invention, the double-stranded molecule produced by the RNA polymerase under microwave irradiation is separated into single strands resulting in an ssRNA and the template. This step may be carried out by heat or chemical denaturation or enzymatically, e.g. by an enzyme capable of separating single-stranded polynucleotides into single-stranded ones such as a helicase. However, according to especially preferred embodiments of the present invention this and other separation steps of double-stranded polynucleotides produced by the RNA polymerase under microwave irradiation as defined herein is carried out by the same enzyme, i.e. the RNA polymerase itself, which step is preferable also carried out under irradiation with microwaves. This step makes beneficial use of the strand-displacement activity of the RNA polymerases of caliciviruses.

It is further preferred that the single strands obtained as outlined above are again (or further) incubated with the RNA polymerase of a calicivurs as defined herein under microwave irradiation and under conditions such that the RNA polymerase synthesizes an RNA strand complementary to each of said single strands. It is evident that the steps of RNA synthesis and strand separation can be repeated one or more times, e.g. about 3 to about 40, preferably about 5 to about 30, more preferably about 10 to about 20 times. According to a further preferred embodiment, the method of the invention comprises a final RNA synthesis step. In particular in cases of repeated cycling of strand separation and RNA synthesis, this method leads to the production of almost pure dsRNA, even if the original template contained a segment (one or more) of DNA or consisted of DNA.

As already outlined above, any further strand separation step may be carried out by heat or chemical denaturation or enzymatically, e.g. by an enzyme capable of separating single-stranded polynucleotides into single-stranded ones such as a helicase. More preferably, however, also the further strand separation steps are carried out by the RNA polymerase of a calicivirus. Therefore, it is evident that the preferred method according to the invention comprising several to a multitude of strand separation and RNA synthesis steps may be carried out in a single batch reaction (especially when using templates that do not require a primer for RNA synthesis by the RNA polymerase as defined herein) requiring only one microwave irradiation of a reaction mixture containing the template, an RNA polymerase of a calicivirus, appropriate buffer (see below) and rNTPs (i.e. rATP, rUTP, rCTP and rGTP, or modified or labelled rNTPs as further outlined below).

According to the present invention, the term “RNA polymerisation conditions” means the conditions, in particular relating to buffer, salt and metal ion (if applicable) conditions that allow the RNA polymerase to synthesise an RNA strand complementary to a template strand. Appropriate buffer, salt, metal ion, reducing agent (if applicable) and other conditions of RNA polymerases are known to the skilled person; see, e.g., WO-A-2007/012329. Thus, the polynucleotide template is typically used in amounts of, e.g. 1 microgram to 4 microgram per 50 microliter reaction volume. The concentration of the ribonucleoside triphosphates (including optional modified or labelled ribonucleoside trisphosphate(s) as further outlined below) is preferably in the range of from 0.1 micromol/l to 1 micromol/l, for example 0.4 micromol/l. The concentration of the RNA polymerase may be for example 1 micromol/l to 10 micromol/l.

Typical buffer conditions are 10 to 80 mM, more preferred 20 to 50 mM HEPES, pH 7.0 to 8.0, 1 to 5 mM, for example 3 mM magnesium acetate, magnesium chloride, manganese acetate or manganese chloride and 1 to 5 mM of a reducing agent, for example DTT.

A typical stop solution contains 2 to 10 mM, preferably 4 to 8 mM ammonium acetate, and 50 to 200 mM, for example 150 mM EDTA.

The length and origin of the single-stranded polynucleotide, e.g. an ssRNA template, is generally not critical. The template may have a naturally occurring or artificial sequence, and the template may be chemically synthesized or derived from diverse sources such as total RNA, mRNA or genomic DNA from eukaryotic, prokaryotic or viral origin, plasmid DNA, cDNA, bacmids or any other sources of RNA and DNA. Double-stranded DNA or RNA needs to be separated into ssDNA or ssRNA, respectively, by heat or microwave irradiation or chemical denaturation prior to serving as a template in the methods of the invention.

The method of the present invention is particularly useful for employing RNA templates to either provide double-stranded RNA products and/or to amplify the ssRNA templates. Particularly preferred embodiments of the invention relate to the provision of short RNA molecules for gene silencing applications, either by antisense technology or RNA interference, also for antisense directed against defined sequences of microRNA or non-coding RNA with the aim to inhibit microRNA-driven RNA interference (antagomirs).

For such applications, the template (preferably RNA) to be used in the method of the present invention has typically a length of 8 to 45 nucleotides such as of 15 to 30 nucleotides, preferably of 21 to 28 nucleotides, more preferably of 21 to 23 nucleotides. The molecules of the latter length are particularly useful for siRNA applications.

It is further contemplated that the RNA polymerase employs modified ribonucleotides during RNA synthesis (including terminal transferase activity) under microwave irradiation (besides the optionally present modified GTP (such as 2′-fluoro-GTP or α-thio-GTP) as defined above). For example, the modification may be a label for detecting the double-stranded RNA synthesis product of the RNA polymerase. Alternatively, also the labelling may carried out for detection of the ssRNA product obtained after strand separation. Labels of use in the present invention comprise fluorophores (such fluoresceine), radioactive groups (e.g. ³²P-labelled ribonucleotides) and partners of specific binding pairs such as biotinylated rNTPs.

In certain embodiments of the invention, the at least one modified ribonucleotide to be incorporated by the RNA polymerase activity into the complementary strand may have a chemical modification (one or more of them) at the ribose, phosphate and/or base moiety. With respect to molecules having an increased stability, especially with respect to RNA degrading enzymes, modifications at the backbone, i.e. the ribose and/or phosphate moieties, are especially preferred.

The chemically modified RNA products of the methods of the present invention preferably have an increased stability as compared to the non-modified ss- or dsRNA analogues.

Preferred examples of ribose-modified ribonucleotides are analogues wherein the 2′—OH group is replaced by a group selected from H, OR, R, halo, SH, SR, NH₂, NHR, NR₂ or CN with R being C₁-C₆ alkyl, alkenyl or alkynyl and halo being F, CI, Br or I. It is clear in the context of the present invention, that the term “modified ribonucleoside triphosphate” or “modified ribonucleotide” also includes 2′-deoxy derivatives which may at several instances also be termed “deoxynucleotides”.

Typical examples of such ribonucleotide analogues with a modified ribose at the 2′ position include 5-aminoallyl-uridine, 2′-amino-2′-deoxy-uridine, 2′-azido-2′-deoxy-uridine, 2′-fluoro-2′-deoxy-guanosine and 2′-O-methyl-5-methyl-uridine.

Examples of ribonucleotides leading to a phosphate backbone modification in the desired double-stranded product are phosphothioate analogues.

According to the present invention, the at least one modified ribonucleotide may also be selected from analogues having a chemical modification at the base moiety. Examples of such analogues include, 6-aza-uridine, 8-aza-adenosine, 5-bromo-uridine, 7-deaza-adenosine, 7-deaza-guanosine, N⁶-methyl-adenosine, 5-methyl-cytidine, pseudo-uridine, and 4-thio-uridine.

The above and other chemically modified ribonucleoside triphosphates are commercially available, for example from Sigma-Aldrich Chemie GmbH, Munich, Germany or Trilink technologies, USA

It is to be understood that also the polynucleotide template may contain one or more modified or labelled nucleotides as outlined above and/or further known in the art. Short templates (e.g. as described above) are usually prepared by chemical synthesis. Other methods for providing the single-stranded polynucleotide templates include enzymatic manipulations, for example reverse transcription of RNA and subsequent degradation of the RNA strand, cutting of larger dsDNA molecules by restriction enzyme(s) and subsequent strand separation by heat or chemical denaturation to form ssDNA, preparation of total cellular RNA, preparation of mRNA and so on.

Preferred reaction volumes range from 20 to 200 microliter, preferably 50 to 100 microliter. Typically, the buffer conditions and other conditions as outlined above are provided by mixing appropriate stock solutions (usually 5× or 10× concentrated), adding the RNA polymerase, the template and double distilled or deionised water (which has been preferably made RNAse and/or DNAse free prior to use) to the desired final reaction volume.

The term “effective amount of microwave energy” is the amount of microwave energy required for the RNA polymerisation of a complementary strand on a single-stranded polynucleotide template or to transfer at least one ribonucleotide to the 3′-end of a single-stranded polynucleotide, respectively, using an RNA polymerase of a calicivurs. The concrete amount of microwave energy for a given template may be determined by the skilled person using routine experimentation and depends particularly on the type and length of the template. As used herein the terms “microwave energy”, “microwave irradiation” or “irradiation with microwaves” or simply “microwaves” are used synonymously and relate to the part of the electromagnetic spectrum comprising wavelengths of about 0.3 to 30 cm, corresponding to a frequency of 1 to 100 gigahertz, which is found between the radio and the infra-red regions of the electromagnetic spectrum. The amount of electromagnetic energy absorbed by a living organism is determined by the dielectric properties of the tissues, cells, and biological molecules.

The generation of the microwave energy for the purposes of the present invention is not critical and can be by any means known to the art. For example, suitable means for applying microwave radiation to reaction compositions according to the invention are microwave ovens which are commercially available from numerous suppliers and routinely form part of the standard equipment in most biological laboratories. Such microwave ovens typically have maximum power levels of from about 500 W to about 1000 W. Even the smallest ovens provide ample levels of microwave irradiation for use in this invention and accordingly, it will be convenient to use lower power settings on ovens in which the output power is adjustable. Thus, according to preferred embodiments of the inventive methods disclosed herein, the composition is irradiated with microwaves having a frequency of from about 1500 MHz to about 3500 MHz and having a power of from about 50 to about 1000 W.

According to preferred embodiments of this invention, lower power settings are also used to time-distribute the applied power over a longer time interval and minimize the potential for localized energy uptake and resulting molecular damage. In an especially preferred embodiment, microwave power is applied to the sample over a series of intervals, with “rest” intervals, in which microwave power is not applied to the sample. Power application intervals and rest intervals will usually range from 1 to 60 seconds each, with power application intervals of from 15 to 60 seconds and rest intervals from 0.5 to 5 seconds being preferred. Most preferably, power will be applied for intervals of about 45 seconds, separated by rest intervals of 1 to 2 seconds.

However, especially depending on the length of the single-stranded polynucleotide template, the irradiation step may be carried out in a single application (interval) of microwave energy of a time period of 1 s to 5 min, more preferably 3 s to 120 s. The latter short time periods are especially useful when templates of shorter length (such as templates for preparing short dsRNAs such as siRNAs) are employed. In particular with respect to the preparation of dsRNA from ssRNA templates, it has been shown according to the invention that the method as described herein enables substantially shorter reaction times compared to incubation times known in the art.

The use of microwave irradiation for providing double-stranded polynucleotide products by RNA polymerases of the caliciviruses is particularly beneficial in the context of preparing double-stranded RNA. As mentioned before, such reactions have hitherto been carried out using longer incubation times at 30 to 42° C., and heat can used to separate double-stranded RNA products. However, under such circumstances the thus-produced dsRNA tends to degrade such that the production of pure dsRNA having the desired length is often difficult to achieve.

In contrast thereto, the use of microwave energy according to the present invention accelerates the polymerization reaction by the RNA polymerase such that the problem of RNA degradation is avoided.

It is to be understood that in a preferred mode of practicing this invention, the above efforts to distribute the applied power over time are to be taken in addition to using “power” settings of the apparatus below maximum. In fact, many commercial microwave ovens maintain a constant magnetron output power at 650-900 W and modulate applied power by varying the duty cycle of the magnetron, a setting of “1” corresponding to a 10% duty cycle and a setting of “9” corresponding to a 90% duty cycle. Most preferably, settings of from 1 to 8, i.e. a magnetron duty cycle of from 10% to 80% at an output power of 700 to 800 W, will be used. That aggregate output energy, corresponding to from about 70 watt-seconds to 35,000 watt-seconds, preferably from 3000 to 3500 watt-seconds per interval, will be applied in the intervals or a single interval, as described above.

The Figures show:

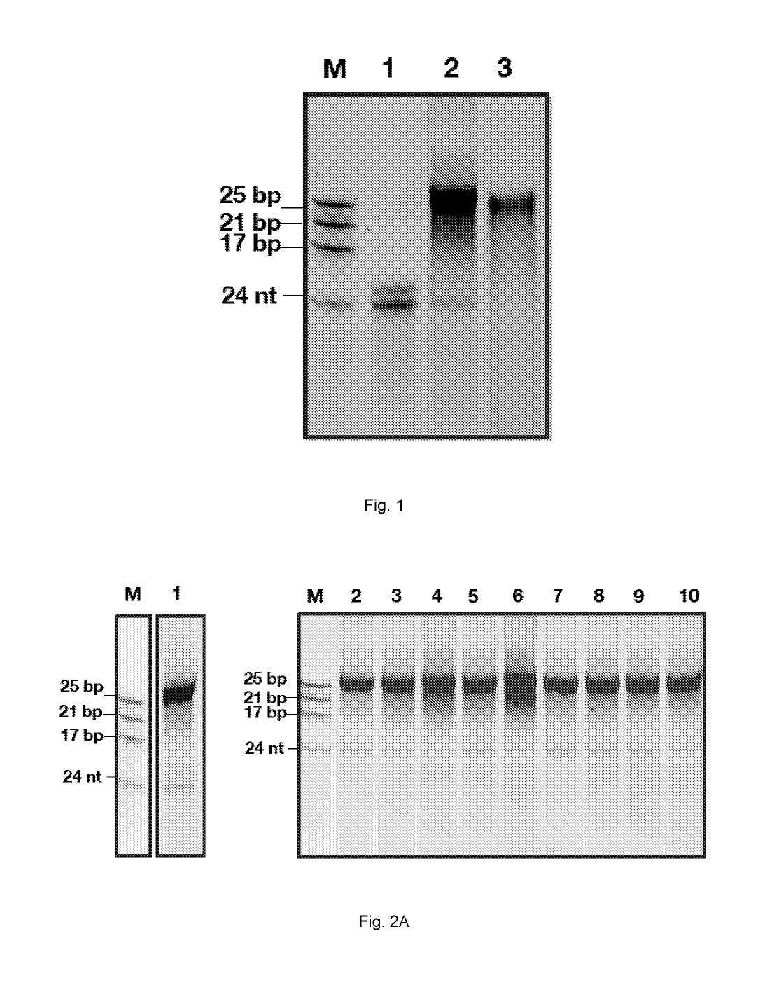

FIG. 1 shows a photograph of an ethidium bromide-stained native 20% polyacrylamide gel after electrophoretic separation of reactions demonstrating the microwave-driven primer-independent de novo initiation of RNA synthesis and generation of a double-stranded RNA by a RNA polymerase of a sapovirus. Lane 1: RNA template. Lane 2: reaction mix of template and RNA polymerase of a sapovirus after microwave irradiation (800 W for 60 s) leads to a band co-migrating with a 25 bp dsRNA marker. Lane 3: product of lane 2 after S1 nuclease treatment. The 25 bp product is not digested with S1 nuclease indicating the double-stranded nature of the product. M: RNA marker corresponding to dsRNA 17 bp, 21 bp and 25 bp and to a ssRNA of 24 nt as indicated.

FIG. 2A shows photographs of ethidium bromide-stained native 20% polyacrylamide gels after electrophoretic separation of reactions used to characterize the energy conditions for microwave-driven primer-independent de novo initiation of RNA synthesis and generation of a double-stranded RNA by a sapovirus RNA polymerase. The sapovirus RNA polymerase was incubated with a 24 nt ssRNA template under microwave irradiation for 60 s at 80 W (lane 1), 160 W (lane 2), 240 W (lane 3), 320 W (lane 4), 400 W (lane 5), 480 W (lane 6), 560 W (lane 7), 640 W (lane 8), 720 W (lane 9) and 800 W (lane 10), respectively. Lanes 1 to 10 each show a product co-migrating with a 25 bp dsRNA marker. M: RNA marker corresponding to dsRNA of 17 bp, 21 bp and 25 bp, respectively, and to a ssRNA of 24 nt, as indicated.

FIG. 2B shows a photograph of an ethidium bromide-stained native 20% polyacrylamide gel after electrophoretic separation of reactions used to characterize the irradiation time needed for microwave-driven primer-independent de novo initiation of RNA synthesis and generation of a double-stranded RNA by a sapovirus RNA polymerase. The sapovirus RNA polymerase was incubated with a 24 nt ssRNA template under microwave irradiation at 80 W for 60 s (lane 1), 30 s (lane 2), 15 s (lane 3) and 5 s (lane 4), respectively, or at 800 W for 60 s (lane 5), 30 s (lane 6), 15 s (lane 7) and 5 s (lane 8), respectively. Each of lanes 1 to 8 shows a product co-migrating with a 25 bp dsRNA marker. M: RNA marker corresponding to dsRNA of 17 bp and 25 bp, respectively and to a ssRNA of 24 nt, as indicated.

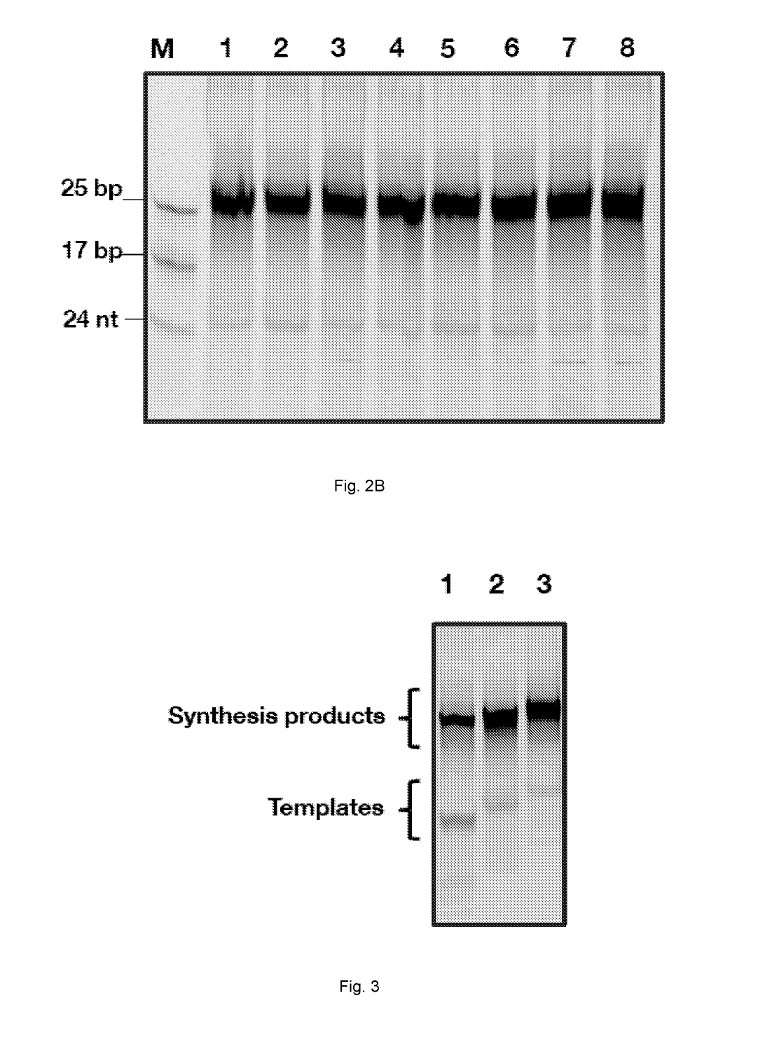

FIG. 3 shows a photograph of an ethidium bromide-stained native 20% polyacrylamide gel after electrophoretic separation of reactions demonstrating that a sapovirus RNA polymerase initiates RNA synthesis on DNA templates de novo in a primer-independent manner and incorporates 2′-fluoro-GMP leading to a double-stranded DNA/RNA product when the reaction is exposed to microwave irradiation. The sapovirus RNA polymerase was incubated with an ssDNA template of 24 nt having 5 dC at the 3′-end (lane 1) or an ssDNA template of the same sequence but having 5 rC at the 3′-end (lane 2) under microwave irradiation at 800 W for 60 s in the presence of rATP, rCTP, rUTP and 2′-fuoro-GTP. A reaction containing the sapovirus RNA polymerase and an ssRNA template of the same sequence as the DNA templates irradiated with microwaves under the same conditions as before and in the presence of rATP, rCTP, rUTP and rGTP served as a control (lane 3). Double-stranded synthesis products and single-stranded templates are indicated.

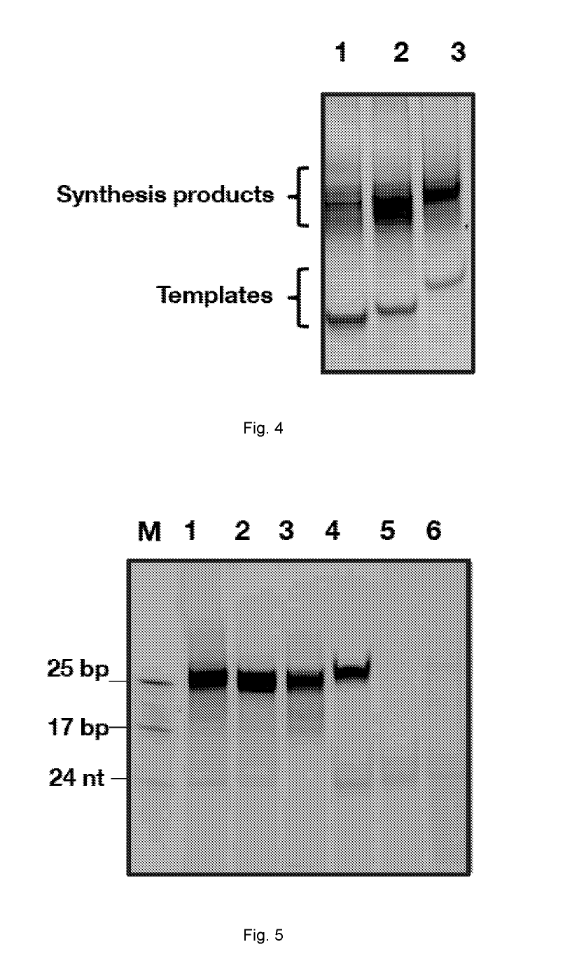

FIG. 4 shows a photograph of an ethidium bromide-stained native 20% polyacrylamide gel after electrophoretic separation of reactions demonstrating that a sapovirus RNA polymerase initiates RNA synthesis on DNA templates de novo in a primer-independent manner and incorporates α-thio-GMP leading to a double-stranded DNA/RNA product when the reaction is exposed to microwave irradiation. The sapovirus RNA polymerase was incubated with an ssDNA template of 24 nt having 5 dC at the 3′-end (lane 1) or an ssDNA template of the same sequence but having 5 rC at the 3′-end (lane 2) under microwave irradiation at 800 W for 60 s in the presence of rATP, rCTP, rUTP and α-thio-GTP. A reaction containing the sapovirus RNA polymerase and an ssRNA template of the same sequence as the DNA templates irradiated with microwaves under the same conditions as before and in the presence of rATP, rCTP, rUTP and rGTP served as a control (lane 3). Double-stranded synthesis products and single-stranded templates are indicated.

FIG. 5 shows a photograph of an ethidium bromide-stained native 20% polyacrylamide gel after electrophoretic separation of reactions demonstrating microwave-driven de novo initiation of RNA synthesis and generation of a double-stranded RNA by RNA polymerases of caliciviruses but not RNA polymerases of other viruses. An ssRNA template of 24 nt was incubated with an RNA polymerase of a sapovirus (lane 1), a norovirus (lane 2), a vesivirus (lane 3), a lagovirus (lane 4), poliovirus (lane 5) or Hepatitis C Virus (lane 6) under microwave irradiation at 800 W for 60 s. Double-stranded products co-migrating with a 25 bp dsRNA marker result with RNA polymerases of caliciviruses (lanes 1 to 4) but not with RNA polymerase of poliovirus (lane 5) or Hepatitis C Virus (lane 6). M: RNA marker corresponding to dsRNA of 17 bp and 25 bp, respectively, and to ssRNA of 24 nt, as indicated.

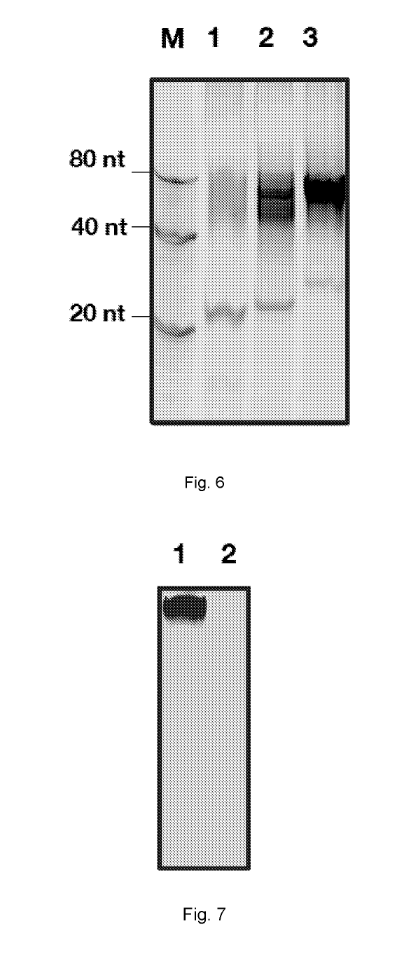

FIG. 6 shows a photograph of an ethidium bromide-stained native 20% polyacrylamide gel after electrophoretic separation of reactions demonstrating microwave-driven de novo initiation of RNA synthesis and generation of a double-stranded DNA/RNA product on ssDNA templates by a sapovirus RNA polymerase. A sapovirus RNA polymerase was incubated with a 24 nt ssDNA template having 5 dC at the 3′-end (lane 1) or a 24 nt ssDNA template of the same sequence but having 5 rC at the 3′-end (lane 3) under microwave irradiation at 800 W for 60 s. As a control, a reaction containing the sapovirus RNA polymerase and an ssRNA template of the same sequence were irradiated with microwaves under the same conditions as before (lane 2). All reactions were carried out in the presence of rATP, rCTP, rUTP and rGTP. A double-stranded product co-migrating with a 25 bp dsRNA marker is visible in lanes 2 and 3, but not in lane 1, indicating that a double-stranded product is generated only in the presence of rC nucleotides at the 3′-end of ssDNA templates. M: RNA marker corresponding to ssDNA of 20 nt, 40 nt and 80 nt, as indicated.

FIG. 7 shows a photograph of an ethidium bromide-stained native 20% polyacrylamide gel after electrophoretic separation of reactions demonstrating that T7 DNA-dependent RNA polymerase is not capable of RNA synthesis under microwave irradiation. The T7 DNA-dependent RNA polymerase was incubated with a dsDNA template (linearized plasmid including 116 bp of an 18 S rRNA sequence) either at 37° C. for 2 h (lane 1) or irradiated with microwaves at 800 W for 60 s (lane 2). All reactions were otherwise carried out under the conditions recommended by the manufacturer of the T7-Megashortscript Kit (Ambion, Inc.). A reaction product is visible after isothermal incubation at 37° C. for 2 h (lane 1) but not after microwave irradiation (lane 2).

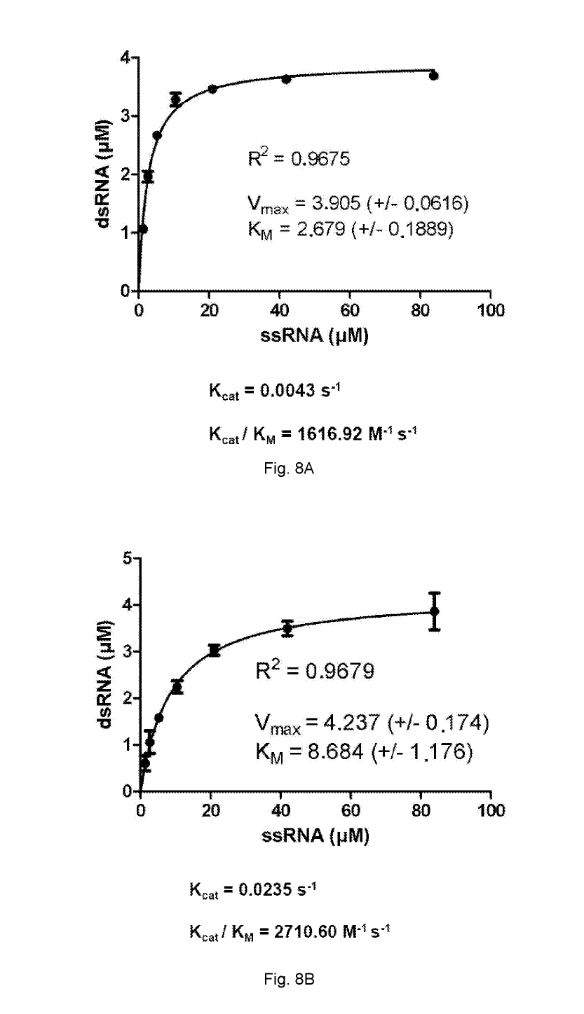

FIG. 8A shows a graph reflecting the kinetics of the enzymatic reaction of primer-independent de novo initiation of RNA synthesis by the sapovirus RdRp using heat generated by a thermoblock. For determination of the reaction kinetics, the sapovirus RdRp (SEQ ID NO: 11) was incubated with an RNA template (5′-AUACCUAGAAUCUGACCAACCCCC-3′; SEQ ID NO: 15). The reactions were performed in a total volume of 25 μl disposed in a thermoblock at 37° C. The reaction mix contained 1 μg of the template, 7.5 μM RdRp, 0.4 mM of each ATP, CTP, UTP, and 2 mM GTP, 5 μl reaction buffer (HEPES 250 mM, MnCl₂ 25 mM, DTT 5 mM, pH 7.6), and RNAse-DNAse free water to a total volume of 25 μl.

FIG. 8B shows a graph reflecting the kinetics of the enzymatic reaction of primer-independent de novo initiation of RNA synthesis by the sapovirus RdRp using microwave energy. For determination of the reaction kinetics, the sapovirus RdRp (SEQ ID NO: 11) was incubated with an RNA template (5′-AUACCUAGAAUCUGACCAACCCCC-3′; SEQ ID NO: 15). The reactions were performed in a total volume of 25 μl disposed in a microwave at 160 Watt. The reaction mix contained 1 μg of the template, 7.5 μM RdRp, 0.4 mM of each ATP, CTP, UTP, and 2 mM GTP, 5 μl reaction buffer (HEPES 250 mM, MnCl₂ 25 mM, DTT 5 mM, pH 7.6), and RNAse-DNAse free water to a total volume of 25 μl.

The present invention is further illustrated by the following non-limiting examples.

### EXAMPLES

### Example 1

### Microwave-Driven Primer-Independent de novo Initiation of RNA Synthesis and Generation of a Double-Stranded RNA by a Sapovirus RNA Polymerase

A sapovirus RNA polymerase (SEQ ID NO: 11) was incubated with an RNA template (5′-AUACCUAGAAUCUGACCAACCCCC-3′; SEQ ID NO: 15) under microwave irradiation at 800 W for 60 s in a conventional microwave oven. The sapovirus RNA polymerase generates a double-stranded RNA (see FIG. 1, lane 2) using the single-stranded RNA as a template. The resulting product was incubated with S1 nuclease. No digestion of the product was observed after incubation with S1 nuclease (see FIG. 1, lane 3), indicating the double-stranded nature of the product. All reactions were performed in a total volume of 25 μl. The RNA polymerisation reaction mix contained 1 μg template, 7.5 μM RNA polymerase, 0.4 mM of each ATP, CTP, UTP, and 2 mM GTP, 5 μl reaction buffer (HEPES 250 mM, MnCl₂ 25 mM, DTT 5 mM, pH 7.6), and RNAse-DNAse free water to a total volume of 25 μl. For S1 nuclease digestion, S1 nuclease (250 U) was added to the reaction and the reaction mix incubated for 1 h at 30° C. The products were separated on a native 20% polyacrylamide gel by electrophoresis and visualized by ethidium bromide staining.

### Example 2

### Characterisation of Microwave Power and Irradiation Time Needed for Microwave-Driven RNA Synthesis by Calicivirus RNA Polymerases

A sapovirus RNA polymerase (SEQ ID NO: 11) was incubated with an RNA template (5′-AUACCUAGAAUCUGACCAACCCCC-3′; SEQ ID NO: 15). The reaction was performed in a total volume of 25 μl disposed in a conventional microwave oven for 60 s at 80 W, 160 W, 240 W, 320 W, 400 W, 480 W, 560 W, 640 W, 720 W and 800 W, respectively. In a further experiment, the reactions were carried out in the same microwave oven at 80 W for 60 s, 30 s, 15 s, or 5 s (see FIG. 2B, lanes 1 to 4), or at 800 W for 60 s, 30 s, 15 s, or 5 s (see FIG. 2B, lanes 5 to 8). A product of the expected size was generated in all reactions (FIG. 2A, 2B).

The reaction mix contained 1 μg template, 7.5 μM RNA polymerase, 0.4 mM of each ATP, CTP, UTP, and 2 mMGTP, 5 μl reaction buffer (HEPES 250 mM, MnCl₂ 25 mM, DTT 5 mM, pH 7.6), and RNAse-DNAse free water to a total volume of 25 μl. The products were separated on a native 20% polyacrylamide gel by electrophoresis and visualized by ethidium bromide staining.

### Example 3

### A Calicivirus RNA Polymerase Initiates RNA Synthesis on DNA Templates de novo in a Primer-Independent Manner and Incorporates 2′-fluoro-GMP Leading to a Double-Stranded DNA/RNA Product When Using Microwave Irradiation

A sapovirus RNA polymerase (SEQ ID NO: 11) was incubated with an ssDNA template (5′-ATACCTAGAATCTGACCAACCCCC-3′; SEQ ID NO: 16) or a DNA template of the same sequence but having a (rC)₅ sequence motif at the 3′-terminus (5′-ATACCTAGAATCTGACCAArCrCrCrCrC-3′; SEQ ID NO: 17). As a control, the sapovirus RNA polymerase was incubated with a single-stranded RNA (5′-AUACCUAGAAUCUGACCAACCCCC-3′; SEQ ID NO: 15) having the same sequence as the above single-stranded DNA. All reactions were performed in a total volume of 25 μl disposed in a conventional microwave oven at 800 W for 60 s. The reaction mix contained 1 μg template, 7.5 μM RNA polymerase, 0.4 mM of each ATP, CTP, UTP, and either GTP (for the control reaction using the ssRNA template; see FIG. 3, lane 3) or 2′-fluoro-GTP (for the reactions using the ssDNA templates; see FIG. 3, lanes 1 and 2), 5 μl reaction buffer (HEPES 250 mM, MnCl₂ 25 mM, DTT 5 mM, pH 7.6), and RNAse-DNAse free water to a total volume of 25 μl. The products were separated on a native 20% polyacrylamide gel by electrophoresis and visualized by ethidium bromide staining.

FIG. 3 (see lanes 1 and 2) demonstrates the microwave-driven RNA synthesis and incorporation of 2′-fluoro-GMP by sapovirus RNA polymerase on ssDNA templates.

### Example 4

### A Calicivirus RNA Polymerase Initiates RNA Synthesis on DNA Templates de novo in a Primer-Independent Manner and Incorporates α-thio-GMP Leading to a Double-Stranded DNA/RNA Product when Using Microwave Irradiation

A sapovirus RNA polymerase (SEQ ID NO: 11) was incubated with an ssDNA template (5′-ATACCTAGAATCTGACCAACCCCC-3′; SEQ ID NO: 16) or a DNA template of the same sequence but having a (rC)₅ sequence motif at the 3′-terminus (5′-ATACCTAGAATCTGACCAArCrCrCrCrC-3′; SEQ ID NO: 17). As a control, the sapovirus RNA polymerase was incubated with a single-stranded RNA (5′-AUACCUAGAAUCUGACCAACCCCC-3′; SEQ ID NO: 15) having the same sequence as the above single-stranded DNA. All reactions were performed in a total volume of 25 μl disposed in a conventional microwave oven at 800 W for 60 s. The reaction mix contained 1 μg template, 7.5 μM RNA polymerase, 0.4 mM of each ATP, CTP, UTP, and either GTP (for the control reaction using the ssRNA template; see FIG. 4, lane 3) or α-thio-GTP (for the reactions using the ssDNA templates; see FIG. 4, lanes 1 and 2), 5 μl reaction buffer (HEPES 250 mM, MnCl2 25 mM, DTT 5 mM, pH 7.6), and RNAse-DNAse free water to a total volume of 25 μl. The products were separated on a native 20% polyacrylamide gel by electrophoresis and visualized by ethidium bromide staining.

FIG. 4 (see lanes 1 and 2) demonstrates the microwave-driven RNA synthesis and incorporation of α-thio-GMP by sapovirus RNA polymerase on ssDNA templates.

### Example 5

### Microwave-Driven Primer-Independent de novo Initiation of RNA Synthesis and Generation of a Double-Stranded RNA by Different RNA Polymerases of Caliciviridae

A sapovirus RNA polymerase (SEQ ID NO: 11), a norovirus RNA polymerase (SEQ ID NO: 9), a vesivirus RNA polymerase (SEQ ID NO: 13) or a lagovirus RNA polymerase (SEQ ID: 14) was incubated under microwave irradiation with an RNA template (5′-AUACCUAGAAUCUGACCAACCCCC-3′; SEQ ID NO: 15). All calicivirus RNA polymerases generate a double-stranded RNA using single-stranded RNA as a template (FIG. 5, lanes 1 to 4). Furthermore, it was investigated whether RNA polymerases of other viral origin (poliovirus, Hepatitis C Virus) were equally able to perform the same reaction under microwave irradiation. Thus, the poliovirus RNA polymerase and the Hepatitis C Virus RNA polymerase were incubated with the same ssRNA template under microwave irradiation. However, the poliovirus and Hepatitis C Virus RNA polymerases do not generate a double-stranded RNA under the same conditions used for the calicivirus RNA polymerases (see FIG. 5, lanes 5 and 6). All reactions were performed in a total volume of 25 μl disposed in a conventional microwave oven at 800 W for 60 s. The reaction mix contained 1 μg template, 7.5 μM RNA polymerase, 0.4 mM of each ATP, CTP, UTP, and 2 mM GTP, 5 μl reaction buffer (HEPES 250 mM, MnCl₂ 25 mM, DTT 5 mM, pH 7.6), and RNAse-DNAse free water to a total volume of 25 μl. The products were separated on a native 20% polyacrylamide gel by electrophoresis and visualized by ethidium bromide staining.

### Example 6

### Microwave-Driven Primer-Independent de novo Initiation of RNA Synthesis and Generation of a DNA-RNA-Double Strand by the Sapovirus RNA Polymerase

The sapovirus RNA polymerase (SEQ ID NO: 11) was incubated with a DNA template (5′-ATACCTAGAATCTGACCAACCCCC-3′; SEQ ID NO: 16) having a (dC)₅ sequence motif at the 3′-terminus, or with a DNA template of the same sequence but having a (rC)₅ sequence motif at the 3′-terminus (5′-ATACCTAGAATCTGACCAArCrCrCrCrC-3′; SEQ ID NO: 17). As a control, the sapovirus RNA polymerase was incubated with a single-stranded RNA displaying the same sequence as the single-stranded DNA templates (5′-AUACCUAGAAUCUGACCAACCCCC-3′; SEQ ID NO: 15). The sapovirus RNA polymerase generates a DNA/RNA double strand using a single stranded DNA as a template (see FIG. 6, lanes 2 and 3) only in the presence of a C ribonucleotide at the 3′-end of the DNA template. All reactions were performed in a total volume of 25 μl disposed in a conventional microwave oven at 800 W for 60 s. The reaction mix contained 1 μg template, 7.5 μM RNA polymerase, 0.4 mM of each ATP, CTP, UTP, and 2 mM GTP, 5 μl reaction buffer (HEPES 250 mM, MnCl₂ 25 mM, DTT 5 mM, pH 7.6), and RNAse-DNAse free water to a total volume of 25 μl. The products were separated on a native 20% polyacrylamide gel by electrophoresis and visualized by ethidium bromide staining.

### Comparative Example

### Comparison of the Efficiency of RNA Synthesis by the T7 DNA-Dependent RNA Polymerase (DdRp) Under Isothermal Conditions and Under Microwave Irradiation

The T7 DdRp was used for RNA synthesis on a double-stranded DNA template (linearized plasmid including a 116 bp of a 18S rRNA sequence, 1 μg pro reaction) under recommended isothermal incubation conditions of 37° C. for 2 h or using microwave irradiation (800 W for 60 s). The reagents of the T7-Megaschortscritp Kit (Ambion, Inc) were used. All reactions were performed according to the manufacturer's instructions. The product of the reaction incubated under isothermal conditions is shown in FIG. 7, lane 1. In comparison, no product was synthesized when the reaction mixture was exposed to microwave irradiation (800 W for 60 s; FIG. 7, lane 2). Reaction products were separated on a native 20% polyacrylamide gel by electrophoresis and visualized by ethidium bromide staining.

The above Examples and Comparative Example show that RNA polymerases of the virus family of Caliciviridae (caliciviruses), but not structurally related RNA-dependent RNA polymerases of other viruses (poliovirus, HCV) or DNA-dependent RNA polymerases (T7 DdRp), polymerise a complementary RNA strand on RNA, DNA or mixed RNA/DNA templates under microwave irradiation.

### Example 7

### Increased Catalytic Efficiency of RNA Synthesis by the Sapovirus RdRp Using Microwave Energy in Comparison to Heat Generated by a Thermoblock

For determination of the reaction kinetics, the sapovirus RdRp (SEQ ID NO: 11) was incubated with an RNA template (5′-AUACCUAGAAUCUGACCAACCCCC-3′; SEQ ID NO: 15). All reactions were performed in a total volume of 25 μl disposed either in a thermoblock at 37° C. or in a microwave oven at 160 Watt. The reaction mix contained 1 μg of the template, 7.5 μM RdRp, 0.4 mM of each ATP, CTP, UTP, and 2 mM GTP, 5 μl reaction buffer (HEPES 250 mM, MnCl₂ 25 mM, DTT 5 mM, pH 7.6), and RNAse-DNAse free water to a total volume of 25 μl. FIG. 8A shows the kinetics of the reaction using heat generated by a thermoblock. FIG. 8B shows the kinetics of the reaction using microwave energy. The median +/−SEM of four independent measures are shown (error bars).

The sapovirus RdRp displays an increased catalytic efficiency (K_{cat} up to 5-fold, and K_{cat}/K_{M} up to 1,6-fold) when using microwave as a source of energy in comparison to heat generated by thermoblock.

1. A method for polymerising a complementary RNA strand on a single-stranded polynucleotide template comprising irradiating a composition containing said template and an RNA polymerase of a virus of the Caliciviridae family under RNA polymerisation conditions, with an effective amount of microwave energy.

2. The method of claim 1 wherein the RNA polymerase is from a virus selected from the group consisting of a norovirus, sapovirus, vesivirus and a lagovirus.

3. The method of claim 1 wherein the RNA polymerase is from a virus selected from the group consisting of a norovirus strain HuCV/NL/Dresden 174/1997/GE (GenBank Acc. No AY741811), an RNA polymerase of the sapovirus strain pJG-Sap01 (GenBank Acc. No AY694184), an RNA polymerase of the vesivirus strain FCV/Dresden/2006/GE (GenBank Acc. No DQ424892), and an RNA polymerase of the lagovirus strain pJG-RHDV-DD06 (GenBank Acc. No. EF363035.1).

4. The method of claim 1 wherein the RNA polymerase has an amino acid sequence selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and SEQ ID NO: 14.

5. The method of claim 1 wherein the composition is irradiated with microwaves having a frequency from 1500 MHz to 3500 MHz and a power from 50 to 1000 W.

6. The method of claim 1 wherein the composition is irradiated with microwaves for 3 s to 120 s.

7. The method of claim 1 wherein the composition comprises at least one modified ribonucleotide.

8. The method of claim 1 wherein the template is selected from the group consisting of single-stranded RNA, single-stranded DNA, and mixed single-stranded DNA/RNA.

9. The method of claim 1 wherein the template has a length from 15 to 30 nucleotides, and the irradiation step is carried out in the absence of a primer.

10. The method of claim 1 wherein the template has at least one C nucleotide at a 3′-end.

11. The method of claim 1 wherein the composition containing the template and the RNA polymerase of a virus of the Caliciviridae family is first irradiated with an effective amount of microwave energy in the presence of rCTP as the only nucleotide before carrying out the irradiation step under RNA polymerisation conditions.

12. The method of claim 1 wherein the RNA polymerase, under microwave irradiation, separates a double-stranded product into single-strands, synthesizes a complementary RNA strand, and repeats the steps of separating complementary strands and RNA synthesis, one or more times.

13. A method for transferring one or more ribonucleotides to a 3′ end of a single-stranded polynucleotide template comprising the step of irradiating a composition containing an RNA polymerase of a virus of the Caliciviridae family in the presence of a nucleotide selected from the group consisting of rATP, rGTP, rUTP, rCTP, and a modified analogue thereof, with an effective amount of microwave energy.

14. The method of claim 13 wherein the template is selected from the group consisting of single-stranded RNA, single-stranded DNA, and mixed single-stranded DNA/RNA.

15. The method of claim 1 wherein the irradiation step is performed in the presence of a primer hybridised to the template.

16. The method of claim 1 wherein said template comprises deoxyribonucleotides at a 3′ end, and the irradiation is carried out in the presence of a modified GTP.

17. The method of claim 16 wherein the modified GTP is selected from the group consisting of 2′-fluoro-GTP and α-thio-GTP.

18. The method of claim 1 wherein the template comprises a deoxyribonucleotide at a 3′ end, wherein the deoxyribonucleotide is not a deoxy-C nucleotide, and the irradiation step is carried out in the presence of a primer hybridised to the template.

19. The method of claim 1 wherein the template comprises polyadenylation, polyguanylation or polyuridylation, and the irradiation step is carried out in the presence of a primer hybridised to the template.

20. The method of claim 1 wherein the template comprises polycytidylation, and the irradation step is carried out in the absence of a primer, wherein the composition comprises a surplus of GTP with respect to other required rNTPs.

21. The method of claim 12 wherein the synthesis step is performed in the presence of a primer.
